(19) United States
(12) Patent Application Publication
(10) Pub. No.: US 2023/0416664 A1
(43) Pub. Date: Dec. 28, 2023

(54) OPEN-WELL MICROCAVITY PLATE

(71) Applicant: CORNING INCORPORATED, Corning, NY (US)

(72) Inventors: William Joseph Lacey, North Andover, MA (US); Allison Jean Tanner, Portsmouth, NH (US)

(21) Appl. No.: 18/037,229

(22) PCT Filed: Nov. 17, 2021
(86) PCT No.: PCT/US2021/059622

§ 371 Date: May 16, 2023

Related U.S. Application Data
(60) Provisional application No. 63/116,280, filed on Nov. 20, 2020.

(51) Int. Cl. C12M 1/12; C12M 1/00

(52) U.S. Cl. CPC C12M 25/06; C12M 27/20

(57) ABSTRACT

Cell culture devices comprise a frame comprising an open well disposed therein and a fluid inlet area in communication with the open well. The open well comprises a top opening, a bottom plate comprising a microcavity substrate, the bottom plate defining a major surface, and one or more sidewalls extending from the bottom plate to the top opening. The microcavity substrate comprises a plurality of microcavities, and each microcavity is configured such that cells cultured in the well form a spheroid. The cell culture device may be a reservoir open well microcavity plate used for spheroid cell culture.

### CROSS-REFERENCE TO RELATED APPLICATIONS

This application claims the benefit of priority under 35 U.S.C. § 119 of U.S. Provisional Application Ser. No. 63/116,280 filed on Nov. 20, 2020, the content of which is relied upon and incorporated herein by reference in its entirety.

### TECHNICAL FIELD

This disclosure generally relates to cell culture apparatus and methods. In particular, the present disclosure relates to open well microcavity plates for use in cell culture.

### BACKGROUND

Cells cultured in a three-dimensional (3D) cell culture environment exhibit more in vivo-like functionality than cells cultured in two-dimensional (2D) environments as monolayers. In 2D cell culture systems, cells attach to a substrate on which they are cultured. In contrast, when grown in 3D systems, cells interact with each other rather than attaching to the substrate to form 3D cell cultures or spheroids.

However, challenges exist when growing 3D cell cultures in a conventional culture apparatus. One difficulty involves maintaining a consistent size and culture environment for spheroids grown in separate wells of a cell culture apparatus. For example, seeding density and growth time may affect repeatability from system to system or from well to well within a given system. As the density of cells grown in a cell culture apparatus increases, larger volumes of cell culture media or more frequent exchange of cell culture media are needed to maintain the cells. Increased frequency of media exchange may create an inconvenience for users and increased volume of cell culture media typically leads to an increased height of media above the cultured cells, resulting in an undesirable decrease in the gas exchange rate for the cells through the media. Furthermore, conventional methods of growing cells in high density as spheroid clusters in wave bags, spinners, and shakers may present inconsistencies in growth and may tend to break up spheroids into smaller clusters. Challenges may also exist when conventional cell culture or tissue culture plates are used for high density spheroid growth, as spheroid clusters are often broken up or disturbed during media exchange.

### SUMMARY

Embodiments of the disclosure provide a microcavity plate for bulk spheroid production that allows for ease of media exchange. A fluid inlet area in the microcavity plate provides a place for a pipette tip during media exchange. By providing an area for the pipette tip, embodiments described herein resolve the existing equipment problems wherein the tip is placed in the culture area and disrupts or dislodges the spheroids. The fluid inlet area also allows for fluid to be introduced to the plate while minimizing sloshing or turbulence. The fluid inlet area may also serve as a fluid outlet area. The microcavity plate comprises an open well with a microcavity substrate bottom portion having shallow microcavities for growing spheroids. When a pipette tip is placed in the fluid inlet area and fluid is introduced to the microcavity plate, turbulence from the entering fluid is minimized and the spheroids are not disrupted or dislodged from the microcavities by the fluid or by the pipette tip.

By providing a microcavity substrate in an open well, embodiments of the disclosure allow spheroids to be cultured together within the same environment from the start of the culture process. Culturing the spheroids together in the same environment allows for consistency of size and growth, while minimizing disruption to the spheroid clusters from media exchange, thereby embodiments described here resolve existing conventional equipment issues.

In an aspect, a cell culture device comprises a frame comprising an open well disposed therein; and a fluid inlet area in communication with the open well. The open well comprises a top opening, a bottom plate comprising a microcavity substrate, the bottom plate defining a major surface, and one or more sidewalls extending from the bottom plate to the top opening.

In some embodiments, the fluid inlet area comprises a face of a sidewall of the one or more sidewalls. In some embodiments, the face of the sidewall is slanted from a top outer portion of the sidewall to a bottom inner portion of the sidewall along the length of the sidewall. In some embodiments, the top outer portion is at a same level as the top opening. In some embodiments, the bottom inner portion is at a same level as the major surface and in communication with the major surface.

In some embodiments, the fluid inlet area comprises a notch disposed in a sidewall of the one or more sidewalls. In some embodiments, the notch comprises a tetrahedron-shaped notch at a center of the sidewall. In some embodiments, an edge of the tetrahedron-shaped notch is slanted from a top outer portion of the sidewall to a bottom inner portion of the sidewall. In some embodiments, the top outer portion is at a same level as the top opening. In some embodiments, the bottom inner portion is at a same level as the major surface and in communication with the major surface.

In some embodiments, the fluid inlet area comprises a notch disposed at a corner of the open well where a first sidewall of the one or more sidewalls joins a second sidewall of the one or more sidewalls at a right angle. In some embodiments, the notch comprises a tetrahedron-shaped notch at the corner of the open well. In some embodiments, an edge of the tetrahedron-shaped notch is slanted from a top outer portion of the corner to a bottom inner portion of the corner. In some embodiments, the top outer portion of the corner is at a same level as the top opening. In some embodiments, the bottom inner portion of the corner is at a same level as the major surface and in communication with the major surface.

In some embodiments, the fluid inlet area comprises a ledge disposed in a sidewall of the one or more sidewalls. In some embodiments, the ledge is a grooved channel. In some embodiments, the ledge slants from a top portion on a first end of the sidewall to a bottom portion on a second end of the sidewall. In some embodiments, the bottom portion is at a same level as the major surface and in communication with the major surface. In some embodiments, the top portion is at a same level as the top opening.

In some embodiments, the fluid inlet area is a fluid outlet area.

In some embodiments, the cell culture device further comprises a baffle. In some embodiments, the baffle is disposed within the open well between the major surface and the top opening. In some embodiments, the baffle comprises a plurality of baffle segments, each baffle segment stretching from one end of the open well to an opposite end of the open well. In some embodiments, at least one baffle segment of the plurality of baffle segments is perpendicular to other baffle segments. In some embodiments, a first baffle segment is disposed in the open well along a length of a sidewall and adjacent to the fluid inlet area.

In some embodiments, the microcavity substrate comprises a plurality of microcavities. In some embodiments, the plurality of microcavities are arranged in at least one row. In some embodiments, the plurality of microcavities are arranged in a hexagonal close-pack pattern.

In some embodiments, each microcavity of the plurality of microcavities comprises a top aperture, a bottom, and a microcavity sidewall surface extending from the top aperture to the microcavity bottom. In some embodiments, the top apertures of the microcavities are co-planar with the major surface and the bottom of the microcavities are positioned below the major surface. In some embodiments, each microcavity comprises a rounded bottom. In some embodiments, a width of the top aperture of each microcavity is 500 μm to 5 mm. In some embodiments, a depth of each microcavity of the plurality of microcavities is 500 μm to 6 mm.

In some embodiments, each microcavity is non-adherent to cells. In some embodiments, an interior surface of each microcavity is coated with an ultra-low-adhesion material. In some embodiments, each microcavity is configured such that cells cultured in the well form a spheroid.

In some embodiments, the one or more sidewalls defines a reservoir above the microcavity substrate. In some embodiments, the one or more sidewalls have a height of 0.780 inches.

In some embodiments, an inner surface of the open well is non-adherent to cells. In some embodiments, the inner surface of the open well comprises a non-adherent surface coating comprising perfluorinated polymers, olefins, agarose, non-ionic hydrogels, polyethers, polyols, polymers that inhibit cell attachment, or a combination thereof. In some embodiments, the non-adherent surface coating comprises an ultra-low attachment (ULA) surface coating.

In some embodiments, the frame, one or more sidewalls, or a combination thereof are formed from polystyrene, polypropylene, polyethylene, polyethylene terephthalate, polymethylpentene, polycarbonate, polymethyl methacrylate, styrene-ethylene-butadiene-styrene, a silicone rubber or copolymer, ethylene vinyl acetate, polysulfone, polytetrafluoroethylene, poly(styrene-butadiene-styrene), or a combination thereof.

In some embodiments, the microcavity substrate is formed from polydimethylsiloxane (PDMS), polymethylpentene, (poly)4-methylpentene (PMP), polyethylene (PE), polystyrene (PS), polypropylene, polyethylene terephthalate, polycarbonate, polymethyl methacrylate, styrene-ethylene-butadiene-styrene, a silicone rubber or copolymer, ethylene vinyl acetate, polysulfone, polytetrafluoroethylene, poly(styrene-butadiene-styrene), or a combination thereof.

In some embodiments, the cell culture device is a reservoir open well microcavity plate.

### BRIEF DESCRIPTION OF THE DRAWINGS

FIG. 1 shows an overhead view of an embodiment of a cell culture device.

FIG. 2 shows a side cross-sectional view of the cell culture device shown in FIG. 1.

FIG. 3 shows an overhead view of an embodiment of a cell culture device.

FIG. 4 shows a close-up view of a feature of the cell culture device shown in FIG. 3.

FIG. 5 shows an overhead view of an embodiment of a cell culture device.

FIG. 6 shows a close-up view of a feature of the cell culture device shown in FIG. 5.

FIG. 7 shows an overhead view of an embodiment of a cell culture device.

FIG. 8 shows a close-up view of a feature of the cell culture device shown in FIG. 7.

FIG. 9 shows a close-up view of an embodiment of a microcavity substrate.

FIG. 10 shows a close-up view of an embodiment of a microcavity substrate.

FIG. 11 shows a close-up view of an embodiment of a microcavity substrate.

### DETAILED DESCRIPTION

Cell responses in 3D cell cultures, such as 3D spheroids or organoids (hereafter referred to as spheroids), are more similar to in vivo behavior than the cell responses in 2D cell cultures, where cells are cultured in a monolayer. The additional dimensionality of 3D cultures is believed to lead to the differences in cellular responses because it influences the spatial organization of the cell surface receptors engaged in interactions with surrounding cells and induces physical constraints to cells, thereby affecting the signal transduction from the outside to the inside of cells, ultimately influencing gene expression and cellular behavior. However, conventional culture devices for generating 3D cell cultures or spheroids suffer from drawbacks due to exchange of media. Spheroids may be dislodged or disrupted from the pipette tip or from the fluid introduced when media is exchanged.

Furthermore, the formation of three-dimensional (3D) cell agglomerates such as spheroids, as opposed to two-dimensional cell culture in which the cells form a monolayer on a surface, increases the density of cells grown in a cell culture apparatus. The increased density of cells in turn increases nutrient demands of the cells cultured in the apparatus. Therefore, media changes are needed more frequently during bulk culture of spheroids.

In some embodiments, provided herein are cell culture devices comprising a frame having an open well disposed therein. The open well comprises a top opening, a microcavity substrate bottom surface, and a sidewall or sidewalls extending from the bottom surface to the top opening. The cell culture devices further comprise a fluid inlet area.

Embodiments described herein provide reservoir microcavity plates having an area separate from the microcavity culture area to add and remove fluid. As described herein, reservoir plates may be referred to as “open-well” plates. In embodiments, the open-well plate comprises an undulating microcavity substrate, where an upper surface and a lower surface of the microcavity substrate undulate together.

Microcavity plates according to embodiments described herein comprise a fluid inlet area. The fluid inlet area is sized for receiving a pipette tip, such as for fluid introduction or for aspiration. The fluid inlet area is in fluid communication with the microcavity substrate. In embodiments, the fluid inlet area may also be a fluid outlet area.

In some embodiments, the fluid inlet area has a bottom spaced away from the microcavity substrate, such as spaced away at a higher elevation or relative altitude. In some embodiments, the fluid inlet area deflects fluid dispensed from a pipette away from the microcavity substrate to avoid disrupting or disturbing the spheroids.

Embodiments of the open well microcavity plates described herein may be covered by a standard microplate lid to decrease likelihood of contaminating the culture. The lid may be lifted off the culture entirely or merely positioned off to the side allowing the area for pipetting to be exposed.

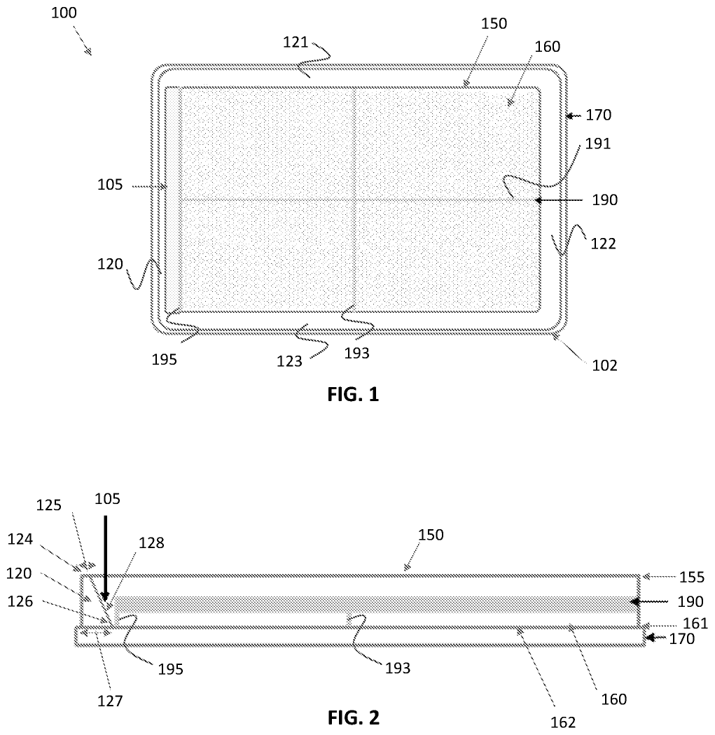

FIG. 1 shows an overhead view of an embodiment of a cell culture device, such as microcavity plate 100. FIG. 2 shows a close-up side cross-sectional view of the microcavity plate 100 of FIG. 1. The cell culture device may comprise a frame 102 comprising an open well 150 disposed therein and a fluid inlet area 105 in communication with the open well 150. The open well 150 comprises a top opening 155, a bottom plate 162 comprising a microcavity substrate 160 defining a major surface 161, and one or more sidewalls 120, 121, 122, 123 extending from the bottom plate 162 to the top opening 155. A skirt 170 may be disposed around a perimeter of the cell culture device, such as to provide stability for the cell culture device.

As shown in FIGS. 1 and 2, the microcavity plate 100 comprises a long fluid inlet/outlet area 105, which runs along the width of the growth surface area. The fluid inlet area 105 comprises a face 128 of a sidewall 120 of the one or more sidewalls 120, 121, 122, 123. The face 128 of the sidewall 120 is slanted from a top outer portion 124 of the sidewall to a bottom inner portion 126 of the sidewall along the length of the sidewall 120. The top outer portion 124 is at a same level as the top opening 155. The bottom inner portion 126 is at a same level as the major surface 161 and in communication with the major surface 161. The major surface 161 is a cell growth area defined by the microcavity substrate 160. The sidewall 120 in which the fluid inlet area is disposed may be described as canted, as a width 125 of the top portion of the sidewall 120 is narrower than a width 127 of the bottom portion of the sidewall 120.

The cell culture device may further include a baffle 190. The baffle 190 may be configured to fit within the open well and may comprise a plurality of baffle segments, such as baffle segment 191, baffle segment 193, and baffle segment 195. As shown in FIG. 1, baffle segment 191 may be disposed along a central length of the open well, extending from sidewall 120 to opposite sidewall 122. Baffle segment 193 and baffle segment 195 may be disposed perpendicular to baffle segment 191, extending along a width of the open well from sidewall 121 to opposite sidewall 123. Baffle segment 193 may be disposed along a central width of the open well. Baffle segment 195 extend along first sidewall 120 and may be disposed parallel to baffle segment 193 at an end of baffle 191 proximate to first sidewall 120. Baffle segment 195 may form one wall of the fluid inlet/outlet area to help keep the pipette tip in place. A user would place a stripette tip (e.g., Corning® Costar® Stripette® serological pipettes) into the fluid inlet/outlet area to remove and dispense fluid during media changes.

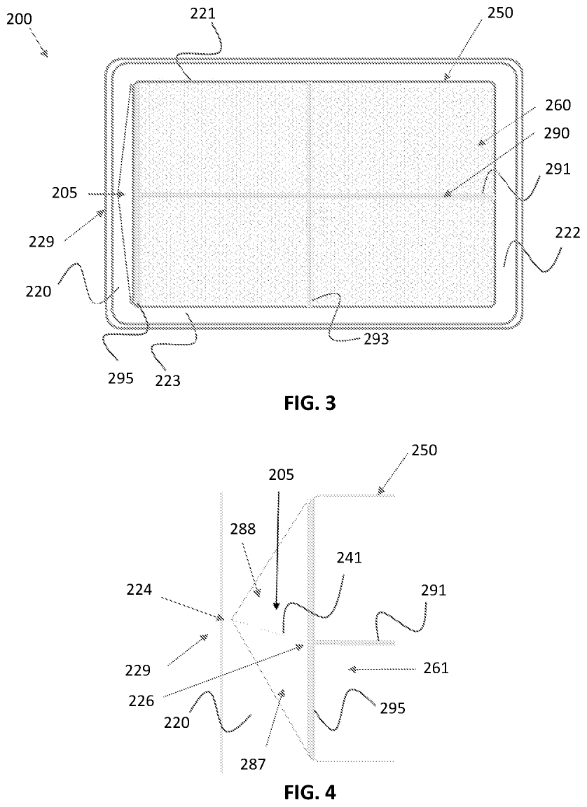

FIG. 3 shows an overhead view of an embodiment of a microcavity plate 200. FIG. 4 shows a close-up view of the fluid inlet area feature of the microcavity plate 200 of FIG. 3. As shown in FIGS. 3 and 4, the microcavity plate 200 comprises a long, angled reservoir or fluid inlet area 205. The long, angled reservoir or fluid inlet area 205 runs along the width of the growth surface area and tapers toward the edges.

The cell culture device may further include a baffle 290 configured to fit within the open well. The baffle 290 may comprise a plurality of baffle segments, such as baffle segment 291, baffle segment 293, and baffle segment 295. As shown in FIG. 3, baffle segment 291 may be disposed along a central length of the open well, extending from sidewall 220 to opposite sidewall 222. Baffle segment 293 and baffle segment 295 may be disposed perpendicular to baffle segment 291, extending along a width of the open well from sidewall 221 to opposite sidewall 223. Baffle segment 293 may be disposed along a central width of the open well. Baffle segment 295 may extend along first sidewall 220, disposed parallel to baffle segment 293 at an end of baffle segment 291 proximate to first sidewall 220. Baffle 295 may make up one wall of the fluid inlet area 205 space to help confine a stripette tip used in fluid addition and removal.

The fluid inlet area 205 comprises a notch disposed in a sidewall 220 of the one or more sidewalls. The surface areas or faces 287, 288 of the notch on the sidewall 220 opposite the baffle 295 are canted toward one another and form an edge 241. Thus, the notch 205 comprises a tetrahedron-shaped notch at a center 229 of the sidewall, where an edge 241 of the tetrahedron-shaped notch is slanted from a top outer portion 224 of the sidewall 220 to a bottom inner portion 226 of the sidewall 220. The top outer portion 224 may be at a same level as the top opening. The bottom inner portion 226 may be at a same level as the major surface 261 and in communication with the major surface 261. The major surface 261 is a cell growth area defined by the microcavity substrate 260.

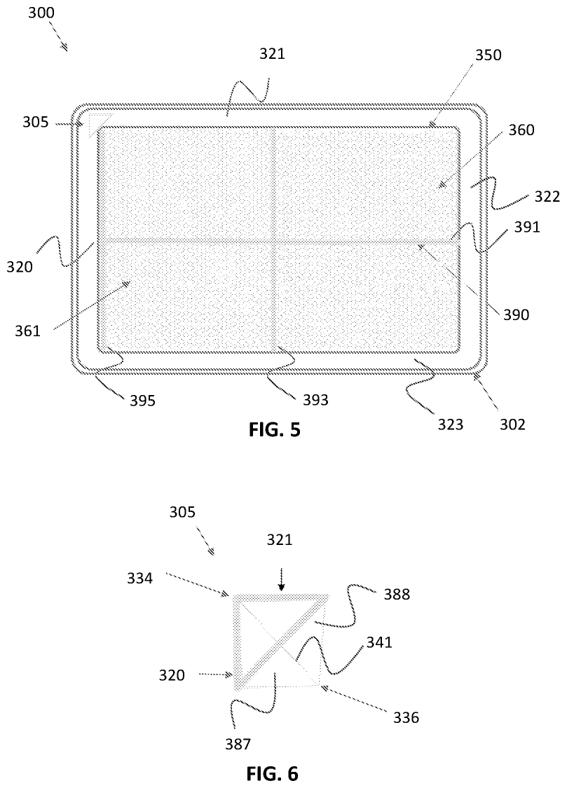

FIG. 5 shows an overhead view of an embodiment of a microcavity plate 300. FIG. 6 shows a close-up view of the fluid inlet area feature of the microcavity plate 300 of FIG. 5. As shown in FIGS. 5 and 6, the microcavity plate 300 comprises a triangular reservoir or notch as a fluid inlet area 305. The notch or triangular reservoir provides an area for a user to place a stripette tip for fluid addition and removal. The fluid inlet area 305 comprises a triangular reservoir or notch disposed at a corner of the open well 350 where a first sidewall 320 of the one or more sidewalls joins a second sidewall 321 of the one or more sidewalls at a right angle. The triangular reservoir lies completely within the boundary of the plate geometry or frame 302 outside the growth surface area except for the very bottom which joins with the growth surface area defined by the microcavity substrate 360. Faces 387, 388 of the sidewalls 320, 321 making up the two outer sides of the triangle are canted toward the area 336 that joins the growth surface area—the first sidewall face 387 and second sidewall face 388 are canted or slanted and form an edge 341 where they meet. Thus, the fluid inlet area 305 comprises a tetrahedron-shaped notch at the corner of the open well 350, where an edge 341 of the tetrahedron-shaped notch is slanted from a top outer portion of the corner 334 to a bottom inner portion of the corner 336. The top outer portion of the corner 334 is at a same level as the top opening. The bottom inner portion of the corner 336 is at a same level as the major surface 361 and in communication with the major surface 361. The major surface 361 is defined by the microcavity substrate 360 and is a cell growth area or cell growth surface.

The cell culture device may further include a baffle 390 configured to fit within the open well. The baffle 390 may comprise a plurality of baffle segments, such as baffle segment 391, baffle segment 393, and baffle segment 395. As shown in FIG. 5, baffle segment 391 may be disposed along a central length of the open well, extending from sidewall 320 to opposite sidewall 322. Baffle segment 393 and baffle segment 395 may be disposed perpendicular to baffle segment 391, extending along a width of the open well from sidewall 321 to opposite sidewall 323. Baffle segment 393 may be disposed along a central width of the open well. Baffle segment 395 may extend along first sidewall 320, disposed parallel to baffle segment 393 at an end of baffle segment 391 proximate to first sidewall 320.

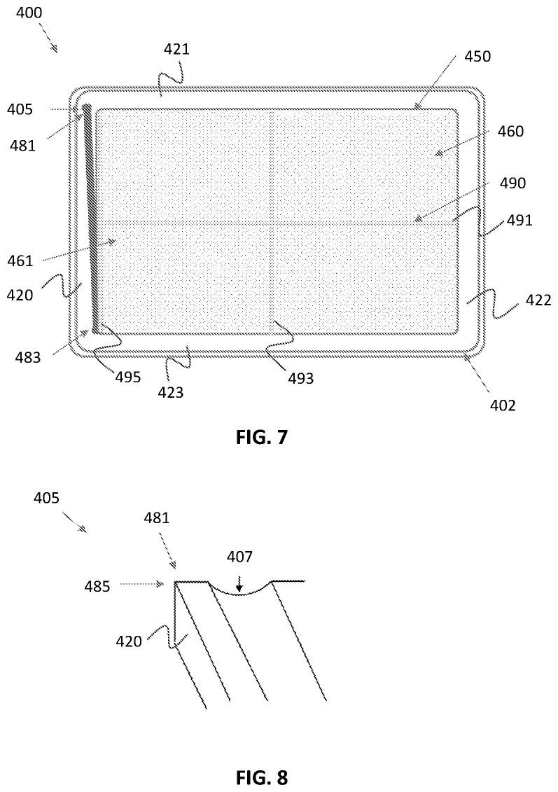

FIG. 7 shows an overhead view of an embodiment of a microcavity plate 400. FIG. 8 shows a close-up view of the fluid inlet area 405 feature of the microcavity plate 400 of FIG. 7. As shown in FIGS. 7 and 8, the microcavity plate 400 comprises a fluid inlet area 405 that runs along the width of the growth surface area of the open well 450. The fluid inlet area 405 comprises a fluid ledge or groove 407 is carved out of the perimeter area of the plate geometry. The fluid inlet area 405 comprises a ledge or grooved channel 407 disposed in a sidewall 420 of the one or more sidewalls 420, 421, 422, 423. The ledge 407 slants from a top portion 485 on a first end 481 of the sidewall 420 to a bottom portion on a second end 483 of the sidewall 420. The bottom portion is at a same level as the major surface 461 and in communication with the major surface 461. The major surface 461 is a cell growth surface defined by the microcavity substrate 460. The top portion 485 is at a same level as the top opening. Thus, the fluid inlet area 405 is higher on the side furthest from the growth surface and becomes lower as it diagonally traverses the width until it ends ˜2 mm above the growth surface. A user would place a stripette tip at the top of the ledge 405 on the first end 481 of the sidewall for fluid addition, and at the bottom of the ledge 405 on the second end 483 of the sidewall for fluid removal.

The cell culture device may further include a baffle 490 configured to fit within the open well. The baffle 490 may comprise a plurality of baffle segments, such as baffle segment 491, baffle segment 493, and baffle segment 495. As shown in FIG. 7, baffle segment 491 may be disposed along a central length of the open well, extending from sidewall 420 to opposite sidewall 422. Baffle segment 493 and baffle segment 495 may be disposed perpendicular to baffle segment 491, extending along a width of the open well from sidewall 421 to opposite sidewall 423. Baffle segment 493 may be disposed along a central width of the open well. Baffle segment 495 may extend along first sidewall 420, disposed parallel to baffle segment 493 at an end of baffle segment 491 proximate to first sidewall 420.

In some embodiments, the cell culture apparatus may include a bottom plate or bottom surface and one or more sidewalls. In some embodiments, cell culture devices herein comprise a bottom plate defining a major surface, one or more sidewalls extending from the bottom plate defining a reservoir, and a plurality of microcavities formed in the major surface. The bottom plate may be formed, in whole or in part, from a substrate having an array of microcavities that promote or induce the growth of spheroids. Each microcavity defines an upper aperture co-planar with the major surface and open to the reservoir, and a microcavity-bottom nadir positioned below the major surface. In contrast to conventional well plates, the plates described herein define a reservoir above the surface of the microcavities, which allows for increased volumes of cell culture media to be used and thus provides for less frequent media exchange. Reservoir plates described herein permit the addition of culture medium in excess of what would be typically used to fill individual shallow wells of a microwell plate and allows cells cultured in different microcavities to be in fluid communication.

In some embodiments, the one or more sidewalls may extend farther away (e.g., a sidewall height) from the bottom plate than some currently available cell culture apparatuses, allowing the reservoir to hold a larger than normal volume of medium. The larger capacity opportunity for the reservoir may allow an excess of culture medium to be added to the reservoir so that the spheroids may not need to rely only on the amount of medium in each individual microcavity. Nutrients and metabolites may be exchanged throughout the cell culture medium because the cell culture medium in the reservoir is in communication with all microcavities in the reservoir. As such, spheroids cultured in embodiments of microcavity plates described herein will not need to be fed (i.e., replacing cell culture medium) as frequently as spheroids growing in standard microplate wells. When feeding is required, the cell culture media can be added at the fluid inlet area to prevent the spheroids from being displaced out of their shallow wells by the fluid movement.

In an embodiment, the reservoir open well microcavity plates described herein may comprise deep perimeter sidewalls (e.g., sidewalls deeper than those in a standard well-plate) to hold a larger than normal volume of medium. For example, a height of the cell culture apparatus, or microcavity plate, may be about 0.780 inches, compared to the standard 96-well or 384-well plate height of 0.560 inches (tolerances of dimensions provided are +/−0.010 inches).

Microcavity substrates according to embodiments described herein comprise a plurality of microcavities. Each microcavity may include an inner cavity with a rounded bottom that is non-adherent to cells. Thus, cell culture devices as described herein facilitate 3D cell culture by allowing cells seeded into the microcavities to self-assemble or attach to one another to form a spheroid in each microcavity. Microcavities may be shallow and permit cell culture medium to cover all of the spheroids in all cavities at once to make manual handling easy.

In an embodiment, the top plane of the microcavities may be recessed to a location close to a bottom of the sidewalls. Individual microcavities may hold a small volume of medium. The individual microcavities may have any suitable dimensions. For example, the diameter or width of individual microcavities may be in a range of about 500 microns to about 5 mm. The depth of individual microcavities may be in a range of about 500 microns to about 6 mm. An excess of culture medium may be added to the reservoir so that the spheroids do not need to rely only on the small amount of medium in the individual microcavities.

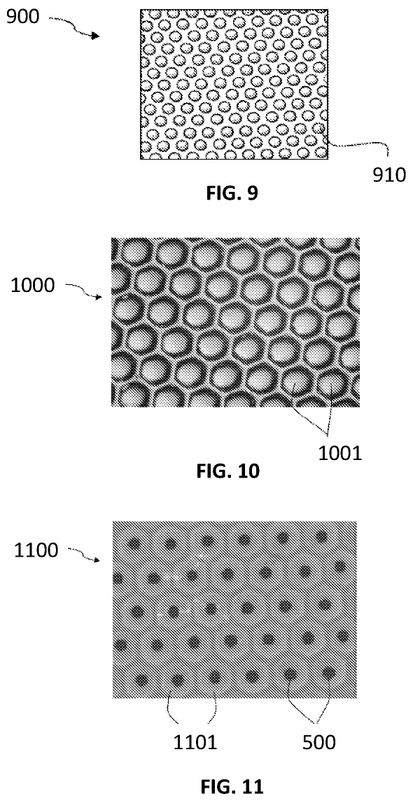

FIG. 9 shows a close-up view of an embodiment of a microcavity substrate patterned with an array of microwells, forming the bottom surface of the open well. The close-up view of the microcavity substrate 900 comprises an array of microcavities 910 or microwells. A structured surface of a cell culture apparatus having an array of microcavities or microwells as described herein may define any suitable number of microcavities that may have any suitable size or shape. The microcavities define a volume based on their size and shape. In many embodiments, one or more or all of the microcavities are symmetric and/or symmetrically rotatable around a longitudinal axis. In some embodiments, the longitudinal axes of one or more or all of the microcavities are parallel with one another. The microcavities may be uniformly or non-uniformly spaced. In some embodiments, the microcavities are uniformly spaced. One or more or all the microcavities can have the same size and shape or can have different sizes and shapes.

In some embodiments, the microcavity substrate defining the microcavities includes an array of hexagonal close-packed microcavities. Such hexagonal close-packing density or “honeycomb” microcavity configuration, combined with the micron-sized geometry of the microcavities, allows for many spheroids to be cultured at once, resulting in bulk spheroid production. An image of an embodiment of such a substrate 1000 is shown in FIG. 10, showing the substrate having an array of hexagonal microcavities 1001. In an embodiment, such packing density allows for approximately 12,588 wells that are 500 μm in diameter in a typical microplate working surface area of about 4.5 inches by about 3 inches. FIG. 11 shows an embodiment of a substrate 1100 where cells (spheroids) 500 are grown in microcavities 1101, and the array of microcavities has a hexagonal close-packed microcavity structure. In some embodiments, the cells within each microcavity 1101 form a single spheroid 500, as depicted.

Microcavity plates according to embodiments of the disclosure provide a homogenous culturing environment. All spheroids cultured in the microcavity plate may receive the same treatment at the same time, thereby providing a homogenous culture environment. In contrast, typical plates with individual wells have more of a heterogenous culture environment because dispensing the same volume to each well is difficult, even with automated equipment.

In certain embodiments, cell culture apparatuses herein comprise a microcavity substrate as a bottom surface of the open well. The microcavity substrate comprises a plurality of microcavities. Each microcavity in the plurality of microcavities may be configured to cause cells cultured in the microcavities to form spheroids of a specified diameter. The microcavities may be any size suitable for culturing spheroids or 3D cell cultures. In some embodiments, the width of the microcavities may be in a range from about 500 microns wide to about 5 mm in width. In some embodiments, the depth of the microcavities may be in a range from about 500 microns deep to about 6 mm deep. For example, in embodiments with the larger size microcavities, the microcavities overlap with spheroid plate well sizes, thereby allowing for organoid development in bulk culture.

In some embodiments, a cell culture device comprises from 8 to about 10,000 microcavities (8, 16, 24, 32, 48, 64, 96, 128, 256, 384, 500, 600, 700, 800, 1000, 1536, 2000, 2400, 3200, 4000, 10000, or any ranges therein). In some embodiments, the plurality of microcavities are arranged in at least one row. In some embodiments, devices comprise multiple rows of microcavities. In some embodiments, the microcavity substrate provides a structured surface defining a plurality of gas permeable wells or microcavities. In some embodiments, the microcavities are in gaseous communication with an exterior of the apparatus via gas permeable materials. In some embodiments, the structured surface defines a plurality of gas permeable microcavities.

Depending on the initial polymer film thickness and process parameters, surfaces with microwells that have different bottom thickness are generated. In some embodiments, polymer thickness of the microwell bottom has a direct impact on oxygen permeability. Thinner microwell bottoms allow better oxygen supply to cells located inside the microwells. The above fabrication method delivers a surface with highly oxygen permeable microwells.

In some embodiments, each of the plurality of microcavities defines a top aperture, a microcavity bottom, and a microcavity sidewall surface extending from the top aperture to the microcavity bottom. The opening or top aperture of the microcavities may have any suitable shape. For example, the opening may be circular, hexagonal, etc. In some embodiments, the bottom of the microcavities comprises a rounded bottom. In some embodiments, the bottom of each microcavity is rounded (e.g., hemispherically round) and sidewalls of the microcavity increase in diameter from the bottom to the top of the microcavity and the boundary between adjacent microcavities is rounded.

In some embodiments, the microcavity shape transitions to alleviate issues with air-escape upon introduction of liquid into the microcavities. In some embodiments, a circular cross-section microcavity bottom (or bottom portion of the microcavity) may be optimal for spheroid formation but problematic for air escape without pocket formation. To alleviate this issue, microcavities may be formed with a circular well-bottom cross-section and a non-circular (e.g., triangular, square, rectangular, pentagonal, hexagonal, etc.) top aperture. In such embodiments, the sidewalls transition from the non-circular (e.g., polygonal) top aperture to the circular microcavity bottom. In some embodiments, the transition is a gradual one, so as to not introduce any interfering, jagged, or horizontal-presenting microcavity sidewall features that could result in the ‘hanging up’ of air bubbles escaping the microcavity upon introduction of liquid to the microcavity. In some embodiments, the corners in the microcavity sidewalls created by the non-circular (e.g., polygonal) shape of the transitioning walls and top aperture provide pathways for the entry of liquid and/or the escape of air.

The microcavity substrate may be formed from the same material or a similar material and method for making the rest of the plate. In some embodiments, the microcavity substrate may be molded or formed separately from the rest of the plate and bonded subsequently through thermal-bonding, ultrasonic welding, or any other method of plastic joining. The material of construction for the microcavity substrate may comprise a plastic polymer, co-polymer, or polymer blend. Nonlimiting examples include silicone rubber, polystyrene, polypropylene, polyethylene, polyethylene terephthalate, polymethylpentene, polycarbonate, polymethyl methacrylate, styrene-ethylene-butadiene-styrene, other such polymers, or a combination thereof. Any suitable construction method may be used to form the microcavity substrate, such as nonlimiting examples include injection molding, thermoforming, 3D printing, or any other method suitable for forming a plastic part.

Microcavity plates according to embodiments described herein may be formed of any suitable material. The material of construction may comprise a plastic polymer, co-polymer, or polymer blend. Nonlimiting examples include silicone rubber, polystyrene, polypropylene, polyethylene, polyethylene terephthalate, polymethylpentene, polycarbonate, polymethyl methacrylate, styrene-ethylene-butadiene-styrene, other such polymers, or a combination thereof. Any suitable construction method may be used to form the microcavity plate. Nonlimiting examples include injection molding, thermoforming, 3D printing, or any other method suitable for forming a plastic part.

In some embodiments, gas-permeable/liquid impermeable materials are used in construction of cell culture devices herein. Any suitable gas-permeable/liquid impermeable materials may be used in embodiments described herein. Nonlimiting examples of gas-permeable/liquid impermeable materials include polystyrene, polycarbonate, ethylene vinyl acetate, polysulfone, polymethylpentene (PMP), polytetrafluoroethylene (PTFE) or compatible fluoropolymer, a silicone rubber or copolymer, poly(styrene-butadiene-styrene), or polyolefin, such as polyethylene or polypropylene, or combinations of these materials. Microcavity substrates may be formed of any suitable material having a suitable gas permeability over at least a portion of the well. Nonlimiting examples of suitable microcavity substrates include polydimethylsiloxane (PDMS), polymethylpentene, (poly)4-methylpentene (PMP), polyethylene (PE), polystyrene (PS), polypropylene, polyethylene terephthalate, polycarbonate, polymethyl methacrylate, styrene-ethylene-butadiene-styrene, a silicone rubber or copolymer, ethylene vinyl acetate, polysulfone, polytetrafluoroethylene, poly(styrene-butadiene-styrene), or a combination thereof. Such materials allow effective gas exchange between the microcavity cell culture area and the outside atmosphere to allow the ingress of the oxygen and other gases, while preventing the passage of liquid or contaminants.

In some embodiments, the thickness of microcavity substrate material is adjusted to allow for optimized gas exchange. The thickness of the microcavity substrate is dependent on the material of construction. In some embodiments, microcavity bottom thickness is between 10 and 75 μm (e.g., 10 μm, 15 μm, 20 μm, 25 μm, 30 μm, 35 μm, 40 μm, 45 μm, 50 μm, 60 μm, 70 μm, 75 μm, and any ranges there between). In embodiments, the microcavities have an oxygen transmission rate through the microcavity substrate gas permeable polymeric material of 2000 cc/m²/day or greater. In some embodiments, the microcavities have a gas permeability through the substrate of 3000 cc/m²/day or greater. In some embodiments, the microcavities have a gas permeability through the substrate of 5000 cc/m²/day or greater.

Cell culture devices described herein allow for generation and culture of 3D cell aggregates. Cells cultured in three dimensions, such as spheroids, can exhibit more in vivo-like functionality than cells cultured in two dimensions as monolayers. In two-dimensional cell culture systems, cells can attach to a substrate on which they are cultured. However, when cells are grown in three dimensions, such as spheroids, the cells interact with each other rather than attaching to the substrate. Cells cultured in three dimensions more closely resemble in vivo tissue in terms of cellular communication and the development of extracellular matrices. Spheroids thus provide a superior model for cell migration, differentiation, survival, and growth and therefore provide better systems for research, diagnostics, and drug efficacy, pharmacology, and toxicity testing.

In some embodiments, the microcavity substrate forms a part of a microcavity plate intended to grow cells or spheroids. The microcavities are structured and arranged to provide an environment that is conducive to the formation of spheroids in culture. That is, in embodiments, the microcavities have spheroid-inducing geometry. For example, the microcavities in which cells are grown can be non-adherent to cells to cause the cells in the microcavities to associate with each other and form spheres. The spheroids expand to size limits imposed by the geometry of the microcavities. In some embodiments, the cell culture substrate in the devices is non-adherent to cells to cause the cells to associate with each other instead of the substrate. For example, in some embodiments, the microcavities are coated with an ultra-low binding material to make the microcavities non-adherent to cells. The combination of non-adherent microcavities, spheroid inducing microcavity geometry, and gravity can define a confinement volume in which growth of cells cultured in the microcavities is limited, which results in the formation of spheroids having dimensions defined by the confinement volume. The spheroids expand to size limits imposed by the geometry of the microcavities. Uniform geometry of the microcavities allows cells grown therein to form similar-sized cell aggregates or spheroids.

In some embodiments, the sidewalls, microcavities, microcavity bottoms, and/or other inner portions of the open well are gas permeable and liquid impermeable. In some embodiments, the sidewalls, microcavities, microcavity bottoms, and/or other inner portions of the open well comprise a low-adhesion or no-adhesion material and/or are coated with a low-adhesion or no-adhesion material. For example, in some embodiments, inside surfaces of the open well are treated with polymers that inhibit cell attachment in order to prevent cell attachment. Nonlimiting examples of such polymers include poly-HEMA, pluronic, or proprietary ULA treatment.

In some embodiments, the inner surface of the microcavities is non-adherent to cells. The microcavities may be formed from non-adherent material or may be coated with non-adherent material to form a non-adherent well. Examples of non-adherent material include perfluorinated polymers, olefins, or like polymers or mixtures thereof. Other examples include agarose, non-ionic hydrogels such as polyacrylamides, polyethers such as polyethylene oxide and polyols such as polyvinyl alcohol, or like materials or mixtures thereof. The combination of, for example, non-adherent wells, well geometry, and gravity can induce cells cultured in the wells to self-assembly into spheroids. Some spheroids can maintain differentiated cell function indicative of a more in vivo like response relative to cells grown in a monolayer.

In some embodiments, the microcavities have a low-binding treatment or are coated with an ultra-low binding material to make the microcavities non-adherent to cells. Examples of non-adherent material include perfluorinated polymers, olefins, or like polymers or mixtures thereof. Other examples include agarose, non-ionic hydrogels such as polyacrylamides, polyethers such as polyethylene oxide and polyols such as polyvinyl alcohol, or like materials or mixtures thereof. The combination of, for example, non-adherent microcavities, microcavities geometry (e.g., size and shape), and/or gravity induce cells cultured in the microcavities to self-assemble into spheroids. Some spheroids maintain differentiated cell function indicative of a more in vivo-like, response relative to cells grown in a monolayer.

In some embodiments, the low-binding treatment or surface coating is a Corning® Ultra Low Attachment (ULA) surface coating. The Corning® ULA surface is hydrophilic, biologically inert and non-degradable, which promotes highly reproducible spheroid formation and easy harvesting. The covalent attachment of Ultra-Low Attachment surface reduces cellular adhesion to the well surface. The Ultra-Low Attachment (ULA) surface allows for uniform and reproducible 3D multicellular spheroid formation.

A wide variety of cell types may be cultured in cell culture devices described herein. For example, any type of cell may be cultured on embodiments of open well microcavity plates described herein including, but not limited to, immortalized cells, primary culture cells, cancer cells, stem cells (e.g., embryonic or induced pluripotent), etc. The cells may be mammalian cells, avian cells, piscine cells, etc. The cells may be in any cultured form including disperse (e.g., freshly seeded), confluent, 2-dimensional, 3-dimensional, spheroid, etc. The cultured cells may further be used in a wide variety of research, diagnostic, drug screening and testing, therapeutic, and industrial applications.

In some embodiments, the cells are mammalian cells (e.g., human, mice, rat, rabbit, dog, cat, cow, pig, chicken, goat, horse, etc.). The cells may be of any tissue type including, but not limited to, kidney, fibroblast, breast, skin, brain, ovary, lung, bone, nerve, muscle, cardiac, colorectal, pancreas, immune (e.g., B cell), blood, etc. Cells may be from or derived from any desired tissue or organ type, including but not limited to, adrenal, bladder, blood vessel, bone, bone marrow, brain, cartilage, cervical, corneal, endometrial, esophageal, gastrointestinal, immune system (e.g., T lymphocytes, B lymphocytes, leukocytes, macrophages, and dendritic cells), liver, lung, lymphatic, muscle (e.g., cardiac muscle), neural, ovarian, pancreatic (e.g., islet cells), pituitary, prostate, renal, salivary, skin, tendon, testicular, and thyroid. In some embodiments, the cell is a somatic cell. In some embodiments, the cell is a stem cell or progenitor cell (e.g., embryonic stem cell, induced pluripotent stem cell) in any desired state of differentiation (e.g., pluripotent, multi-potent, fate determined, immortalized, etc.). In some embodiments, the cell is a disease cell or disease model cell. For example, in some embodiments, the spheroid comprises one or more types of cancer cells or cells that can be induced into a hyper-proliferative state (e.g., transformed cells).

In some embodiments, the systems, devices, and methods herein comprise one or more cells. In some embodiments, the cells are cryopreserved. In some embodiments, the cells are in three-dimensional culture. In some such embodiments, the systems, devices, and methods comprise one or more spheroids. In some embodiments, one or more of the cells are actively dividing. In some embodiments, a spheroid contains a single cell type. In some embodiments, a spheroid contains more than one cell type. In some embodiments, where more than one spheroid is grown, each spheroid is of the same type, while in other embodiments, two or more different types of spheroids are grown. Cells grown in spheroids may be natural cells or altered cells (e.g., cell comprising one or more non-natural genetic alterations).

Any cell culture medium capable of supporting the growth of cells may be used when culturing cells using cell culture devices described in embodiments herein. Cell culture medium may be for example, but is not limited to, sugars, salts, amino acids, serum (e.g., fetal bovine serum), antibiotics, growth factors, differentiation factors, colorant, or other desired factors. Exemplary cell culture medium includes Dulbecco's Modified Eagle Medium (DMEM), Ham's F12 Nutrient Mixture, Minimum Essential Media (MEM), RPMI Medium, Iscove's Modified Dulbecco's Medium (IMDM), MesenCult™-XF medium (commercially available from STEMCELL Technologies Inc.), and the like.

In some embodiments, the systems, devices, and methods comprise culture media (e.g., comprising nutrients (e.g., proteins, peptides, amino acids), energy (e.g., carbohydrates), essential metals and minerals (e.g., calcium, magnesium, iron, phosphates, sulphates), buffering agents (e.g., phosphates, acetates), indicators for pH change (e.g., phenol red, bromo-cresol purple), selective agents (e.g., chemicals, antimicrobial agents), etc.). In some embodiments, one or more test compounds (e.g., drug) are included in the systems, devices, and methods.

Methods for culturing cells on embodiments of open well microcavity plates described herein are also disclosed. In some embodiments, methods comprise cell culture of cell aggregates, or spheroids, in a microcavity plate. Methods of culturing cells using the microcavity plates described herein comprise seeding cells in the microcavity plate. Seeding cells on a microcavity plate may include contacting the plate with a solution containing the cells. Culturing cells on microcavity plate may further include contacting the microcavity plate with cell culture medium. Generally, contacting the microcavity plate with cell culture medium includes seeding or placing cells to be cultured on the microcavity plate in an environment with medium in which the cells are to be cultured. Contacting the microcavity plate with cell culture medium may include pipetting cell culture medium onto the microcavity plate.

In some embodiments, provided herein are methods of culturing spheroids, comprising introducing culture media to a cell culture device described herein and adding spheroid forming cells to the culture media. In some embodiments, methods further comprise replacing or exchanging media (e.g., daily, etc.). For example, cell culture medium may be disposed in the plate for a predetermined period of time. At least some of the cell culture medium may be removed after the predetermined period of time, and fresh cell culture medium may be added. Cell culture medium may be removed and replaced according to any predetermined schedule. For example, at least some of the cell culture medium may be removed and replaced every hour, or every 12 hours, or every 24 hours, or every 2 days, or every 3 days, or every 4 days, or every 5 days.

The cell culture apparatuses described herein can be used to culture cells within microcavities of the apparatus in any suitable manner. For example, a method for culturing cells involves introducing cells and a cell culture medium into one or more of the plurality of microcavities of a cell culture apparatus as described herein. Culturing the cells in one or more of the plurality of microcavities may include forming a spheroid within the one or more microcavities. The spheroid cultured within the one or more microcavities may be defined by a diameter of about, e.g., less than or equal to 500 μm, less than or equal to 400 μm, less than or equal to 300 μm, less than or equal to 250 μm, less than or equal to 150 μm, etc. or any range within the aforementioned values. The diameter of one spheroid may differ from an average diameter of all the spheroids grown in the plurality of microcavities by about, e.g., less than or equal to 20%, less than or equal to 15%, less than or equal to 10%, less than or equal to 5%, less than or equal to 2%, etc. or any range within the aforementioned values.

The open well configuration allows for ease of manual handling and maintenance of an initially homogenous culture environment. The fluid inlet area allows for prevention of spheroid disruption due to the presence of the pipette tip or due to turbulence from media exchange. The cell culture medium may be replaced or exchanged as needed. The pipette may be used to introduce and remove the media from the microcavity plate. The pipette tip may be placed in the fluid inlet area to add cell culture media.

Once the culture has formed the required characteristics, such as number of cells or spheroids, differentiated state, etc., the cell culture medium in the plate may be removed. The pipette tip may be placed in the fluid inlet area to remove cell culture media. In some embodiments, cell culture medium may be removed for the most part, as some cell culture medium may remain in the individual microcavities with the spheroids.

Methods of the disclosure may further comprise harvesting the spheroids. The spheroids may be harvested in any suitable manner. For example, spheroids may be aspirated for removal from the microcavity plate. As another example, gravity may be used to harvest the spheroids from the microcavity plate. For example, in embodiments where the wells are non-adherent to cells, the cells may be harvested by inverting the apparatus to allow gravity to displace the cells from the wells. Other nonlimiting harvest methods include scraping, vibration, and chemical means.

It will be appreciated that the various disclosed embodiments may involve particular features, elements, or steps that are described in connection with that particular embodiment. It will also be appreciated that a particular feature, element, or step, although described in relation to one particular embodiment, may be interchanged or combined with alternate embodiments in various non-illustrated combinations or permutations.

It is also to be understood that, as used herein the terms “the,” “a,” or “an,” mean “at least one,” and should not be limited to “only one” unless explicitly indicated to the contrary. Thus, for example, reference to “an opening” includes examples having two or more such “openings” unless the context clearly indicates otherwise.

All scientific and technical terms used herein have meanings commonly used in the art unless otherwise specified. The definitions provided herein are to facilitate understanding of certain terms used frequently herein and are not meant to limit the scope of the present disclosure.

As used herein, “have,” “having,” “include,” “including,” “comprise,” “comprising,” or the like are used in their open-ended sense, and generally mean “including, but not limited to.”

Ranges may be expressed herein as from “about” one particular value, and/or to “about” another particular value. When such a range is expressed, examples include from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

All numerical values expressed herein are to be interpreted as including “about,” whether or not so stated, unless expressly indicated otherwise. It is further understood, however, that each numerical value recited is precisely contemplated as well, regardless of whether it is expressed as “about” that value. Thus, “a dimension less than 10 mm” and “a dimension less than about 10 mm” both include embodiments of “a dimension less than about 10 mm” as well as “a dimension less than 10 mm.”

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred.

While various features, elements or steps of particular embodiments may be disclosed using the transitional phrase “comprising,” it is to be understood that alternative embodiments, including those that may be described using the transitional phrases “consisting” or “consisting essentially of,” are implied. Thus, for example, implied alternative embodiments to a method comprising A+B+C include embodiments where a method consists of A+B+C, and embodiments where a method consists essentially of A+B+C.

Although multiple embodiments of the present disclosure have been described in the Detailed Description, it should be understood that the disclosure is not limited to the disclosed embodiments, but is capable of numerous rearrangements, modifications and substitutions without departing from the disclosure as set forth and defined by the following claims.

What is claimed is:

1. A cell culture device comprising:
a frame comprising:
an open well disposed therein, the open well comprising:
a top opening,
a bottom plate comprising a microcavity substrate, the bottom plate defining a major surface, and
one or more sidewalls extending from the bottom plate to the top opening; and
a fluid inlet area in communication with the open well.

2. The cell culture device of claim 1, wherein the fluid inlet area comprises a face of a sidewall of the one or more sidewalls.

3. The cell culture device of claim 2, wherein the face of the sidewall is slanted from a top outer portion of the sidewall to a bottom inner portion of the sidewall along the length of the sidewall.

4. The cell culture device of claim 3, wherein the top outer portion is at a same level as the top opening.

5. The cell culture device of claim 3, wherein the bottom inner portion is at a same level as the major surface and in communication with the major surface.

6. The cell culture device of claim 1, wherein the fluid inlet area comprises a notch disposed in a sidewall of the one or more sidewalls.

7. The cell culture device of claim 6, wherein the notch comprises a tetrahedron-shaped notch at a center of the sidewall.

8. The cell culture device of claim 7, wherein an edge of the tetrahedron-shaped notch is slanted from a top outer portion of the sidewall to a bottom inner portion of the sidewall.

9. The cell culture device of claim 8, wherein the top outer portion is at a same level as the top opening.

10. The cell culture device of claim 8, wherein the bottom inner portion is at a same level as the major surface and in communication with the major surface.

11. The cell culture device of claim 1, wherein the fluid inlet area comprises a notch disposed at a corner of the open well where a first sidewall of the one or more sidewalls joins a second sidewall of the one or more sidewalls at a right angle.

12. The cell culture device of claim 11, wherein the notch comprises a tetrahedron-shaped notch at the corner of the open well.

13. The cell culture device of claim 12, wherein an edge of the tetrahedron-shaped notch is slanted from a top outer portion of the corner to a bottom inner portion of the corner.

14. The cell culture device of claim 13, wherein the top outer portion of the corner is at a same level as the top opening.

15. The cell culture device of claim 13, wherein the bottom inner portion of the corner is at a same level as the major surface and in communication with the major surface.

16. The cell culture device of claim 1, wherein the fluid inlet area comprises a ledge disposed in a sidewall of the one or more sidewalls.

17. The cell culture device of claim 16, wherein the ledge is a grooved channel.

18. The cell culture device of claim 16, wherein the ledge slants from a top portion on a first end of the sidewall to a bottom portion on a second end of the sidewall.

19. The cell culture device of claim 18, wherein the bottom portion is at a same level as the major surface and in communication with the major surface.

20. The cell culture device of claim 18, wherein the top portion is at a same level as the top opening.

21. The cell culture device of claim 1, wherein the fluid inlet area is a fluid outlet area.

22. The cell culture device of claim 1, wherein the cell culture device further comprises a baffle.

23. The cell culture device of claim 22, wherein the baffle is disposed within the open well between the major surface and the top opening.

24. The cell culture device of claim 22, wherein the baffle comprises a plurality of baffle segments, each baffle segment stretching from one end of the open well to an opposite end of the open well.

25. The cell culture device of claim 24, wherein at least one baffle segment of the plurality of baffle segments is perpendicular to other baffle segments.

26. The cell culture device of claim 24, wherein a first baffle segment is disposed in the open well along a length of a sidewall and adjacent to the fluid inlet area.

27. The cell culture device of claim 1, wherein the microcavity substrate comprises a plurality of microcavities.

28. The cell culture device of claim 27, wherein the plurality of microcavities are arranged in at least one row.

29. The cell culture device of claim 27, wherein the plurality of microcavities are arranged in a hexagonal close-pack pattern.

30. The cell culture device of claim 27, wherein each microcavity of the plurality of microcavities comprises a top aperture, a bottom, and a microcavity sidewall surface extending from the top aperture to the microcavity bottom.

31. The cell culture device of claim 30, wherein the top apertures of the microcavities are co-planar with the major surface and the bottom of the microcavities are positioned below the major surface.

32. The cell culture device of claim 30, wherein each microcavity comprises a rounded bottom.

33. The cell culture device of claim 30, wherein a width of the top aperture of each microcavity is 500 nm to 5 mm.

34. The cell culture device of claim 30, wherein a depth of each microcavity of the plurality of microcavities is 500 nm to 6 mm.

35. The cell culture device of claim 30, wherein each microcavity is non-adherent to cells.

36. The cell culture device of claim 35, wherein an interior surface of each microcavity is coated with an ultra-low-adhesion material.

37. The cell culture device of claim 30, wherein each microcavity is configured such that cells cultured in the well form a spheroid.

38. The cell culture device of claim 1, wherein the one or more sidewalls defines a reservoir above the microcavity substrate.

39. The cell culture device of claim 38, wherein the one or more sidewalls have a height of 0.780 inches.

40. The cell culture device of claim 1, wherein an inner surface of the open well is non-adherent to cells.

41. The cell culture device of claim 40, wherein the inner surface of the open well comprises a non-adherent surface coating comprising perfluorinated polymers, olefins, agarose, non-ionic hydrogels, polyethers, polyols, polymers that inhibit cell attachment, or a combination thereof.

42. The cell culture device of claim 41, wherein the non-adherent surface coating comprises an ultra-low attachment (ULA) surface coating.

43. The cell culture device of claim 1, wherein the frame, one or more sidewalls, or a combination thereof are formed from polystyrene, polypropylene, polyethylene, polyethylene terephthalate, polymethylpentene, polycarbonate, polymethyl methacrylate, styrene-ethylene-butadiene-styrene, a silicone rubber or copolymer, ethylene vinyl acetate, polysulfone, polytetrafluoroethylene, poly(styrene-butadiene-styrene), or a combination thereof.

44. The cell culture device of claim 1, wherein the microcavity substrate is formed from polydimethylsiloxane (PDMS), polymethylpentene, (poly)4-methylpentene (PMP), polyethylene (PE), polystyrene (PS), polypropylene, polyethylene terephthalate, polycarbonate, polymethyl methacrylate, styrene-ethylene-butadiene-styrene, a silicone rubber or copolymer, ethylene vinyl acetate, polysulfone, polytetrafluoroethylene, poly(styrene-butadiene-styrene), or a combination thereof.

45. The cell culture device of claim 1, wherein the cell culture device is a reservoir open well microcavity plate.
